(19) United States
(12) Patent Application Publication
(10) Pub. No.: US 2006/0002936 A1
(43) Pub. Date: Jan. 5, 2006

(54) Method of modulating platelet activation

(76) Inventors: Jonathan M. Gibbins, Reading (GB); Peter A. Jordan, Reading (GB)

Correspondence Address: FOLEY & LARDNER LLP, 111 HUNTINGTON AVENUE, 26TH FLOOR, BOSTON, MA, 02199-7610, US

(21) Appl. No.: 10/883,169

(22) Filed: Jul. 1, 2004

(51) Int. Cl. A61K 39/395

(52) U.S. Cl. 424/146.1

(57) ABSTRACT

The present invention relates to a method of treating a disease or condition mediated by platelet activation comprising supplying a therapeutically effective amount of an ERP5 inhibitor to a patient in need thereof, so as to reduce the effects of platelet activation in said patient.

### FIELD OF THE INVENTION

The present invention relates to the control of platelet activation, and in particular to control of platelet activation mediated by the thiol isomerase ERP5.

### BACKGROUND TO THE INVENTION

Platelets play an essential role in haemostasis and are essential in the prevention of excessive bleeding. However, changes in their regulation can lead to thrombotic disorders or to conditions characterised by failure of the blood to clot.

In classical terms thiol isomeases have been associated with the formation of a disulphide bond between two cysteine residues through an oxidation reaction. Generally, the reduction/oxidation systems within a cell have been represented very simply. The cytoplasmic environment is hypoxic, and reducing in nature, whereas the extracellular environment is normoxic, and oxidising. Therefore, to correctly generate these bonds inside the cell there are a group of enzymes known as the thiol isomerases. These are capable of the formation, reduction, and rearrangement of the disulphide bonding patterns of proteins, often as part of folding of nascent proteins. The thiol isomerase enzymes are anchored to the endoplasmic reticulum via the KDEL-receptor proteins (Martire G, Mottola G, Pascale M C, et al. Different fate of a single reported protein containing KDEL or KDXX targeting signals stably expressed in mammalian cells. J. Biol. Chem. 1996; 271:3541-3547, Ferrari DM, Soling H-D. The protein disulphide isomerase family: Unraveling a string of folds. Biochem. J. 1999; 339:1-10, Teasdale R D, Jackson M R Signal-mediated sorting of membrane proteins between the endoplasmic reticulum and the golgi apparatus. Annu. Rev. Cell Dev. Biol. 1996; 12:27-54). However, recent studies have suggested an additional degree of functionality for these thiol isomerase enzymes on the extracellular membrane surface of cells, where they can play a role in receptor activation and remodelling, and substrate processing (Essex D W, Li M. Protein disulfide isomerase mediates platelet aggregation and secretion. Br. J. Haematology. 1999; 104:448-454, Tager M, Kroning H, Thiel U, Ansorge S. Membrane-bound protein disulphide isomerase (PDI) is involved in regulation of surface expression of thiols and drug sensitivity of B-CLL cells. Experimental Hematology. 1997; 25:601-607, Ryser H J, Levy E M, Mandel R, DiSciullo G J. Inhibition of Human Immunodeficiency Virus Infection by Agents that Interfere with Thiol-Disulfide Interchange Upon Virus-Receptor Interaction. Proc. Nat'l Acad. Sci., USA. 1994; 91:4559-4563).

Protein disulphide isomerase (PDI) is the best characterised thiol isomerase to demonstrate this dual functionality. A number of cell types including bovine aortic endothelial cells (Hotchkiss K A, Matthias L J, Hogg P J. Exposure of the cryptic Arg-Gly-Asp sequence in thrombospondin-1 by protein disulfide isomerase. Biochim. Biophys. Acta. 1998; 1388:478-488), rat hepatocytes (Terada K, Manchikalapudi P, Noiva R, Jauregui H O, Stockert R J, Schilsky M L. Secretion, surface localisation, turnover, and steady state expression of protein disulfide isomerase in rat hepatocytes. J. Biol. Chem. 1995; 270:20410-20416, Akagi S, Yamamoto A, Yoshimori T, Masaki R, Ogawa R, Tashiro Y. Distribution of protein disulfide isomerase in rat hepatocytes. J. Histochemistry and Cytochemistry. 1988; 36:1533-1542), and human B cells (Tager M et al, supra, Kroning H, Kahne T, Ittenson A, Franke A, Ansorge S. Thiol-proteindisulfide-oxidoreductase (protein disulfide isomerase): A new plasma membrane constituent of mature human B lymphocytes. Scand. J. immunol. 1994; 39:346-350), have been shown to secrete PDI, which associates with the cell surface. Cell-surface PDI has been implicated in the reduction of the disulphide linked diptheria toxin heterodimer (Mandel R, Ryser HJ-P, Ghani F, Wu M, Peak D. Inhibition of a reductive function of the plasma membrane by bacitracin and antibodies against protein disulphide isomerase. Proc. Natl. Acad. Sci. USA. 1993; 90:4112-4116) and events that trigger entry of the human immunodeficiency virus into lymphoid cells (Ryser HJ et al, supra). Based upon a series of investigations, initially by Detweiller and co-workers, a role for PDI in platelet physiology is now established (Essex D W et al, supra, Essex D W, Chen K, Swiatkowska M. Localisation of protein disulfide isomerase to the external surface of the platelet plasma membrane. Blood. 1995; 86:2168-2173, Chen K, Detwiler T C, Essex D W. Characterisation of protein disulphide isomerase released from activated platelets. Br. J. Haematol. 1995; 90:425-431, Chen K, Lin Y, Detwiler T C. Protein disulfide isomerase activity is released by activated platelets. Blood. 1992; 79:2226-2228). Early studies demonstrated that PDI was present on the external membrane of activated and resting platelets and that proteins with thiol isomerase activity were secreted from activated platelets. Indeed, cell-surface exposure of free thiol groups, and those from PDI in particular, are elevated following platelet activation (Burgess J K, Hotchkiss K A, Suter C, et al. Physical proximity and functional association of glycoprotein 1ba and protein disulfide isomerase on the platelet plasma membrane. J. Biol. Chem. 2000; 275:9758-9766). Further studies have demonstrated that inhibition of PDI with specific inhibitory antibodies can block a number of platelet responses to agonists; including, aggregation, adhesion, fibrinogen binding, and integrin activation (Essex D W, Li M, Miller A, Feinman R D. Protien disulfide isomerase and sulfhydryl-dependent pathways in platelet activation. Biochemistry. 2001; 40:6070-6075, Lahav J, Jurk K, Hess O, et al. Sustained integrin ligation involves extracellular free sulfhdryls and enzymatically catalyzed disulfide exchange. Blood. 2002; 100:2472-2478, Lahav J, Wijnen E M, Hess O, et al. Enzymatically catalyzed disuflide exchange is required for platelet adhesion to collagen via integrin a₂b₁. Blood. 2003; 102:2085-2092, Lahav J, Gofer-Dadosh N, Luboshitz J, Hess O, Shaklai M. Protein disulfide isomerase mediates integrin-dependent adhesion. FEBS. 2000; 475:89-92). In addition, reagents that block cell-surface thiol groups such as para-chloromercuriphenyl sulfonate, dithiobisnitrobenzoic acid, and bacitracin have also been shown to inhibit these functions. This inhibition has often been to a greater degree than that observed for anti-PDI antibodies indicating that there may be additional surface proteins involved in this process (Lahav J, Jurk K, et al, supra, Zai A, Rudd A, Scribner A W, Loscalzo J. Cell-surface protein disulfide isomerase catalyses transnitrosation and regulates intracellular transfer of nitric oxide. J. Clin. Invest. 1999; 103:393-399). The mechanistic basis for these observations has not been determined, although it has been proposed that they are based upon an interaction with integrins, and in particular integrins α₂β₁ and α_{IIb}β₃ (Essex D W, Li M et al, supra, Lahav J, Wijnen E M et al, supra). Studies have shown that the different affinity states for the ecto-domain of α_{IIb}β₃ have different conformations and evidence indicates switching between states is a redox active process with a different arrangement of disulphide bonds in the two conformations (Yan B, Smith J W. A redox site involved in integrin activation. J. Biol. Chem. 2000; 275:39964-39972, Jiang X-M, Fitzgerald M, Grant C M, Hogg P J. Redox control of exofacial protein thiols/disulfides by protein disuflide isomerase. J. Biol. Chem. 1999; 274:2416-2423). It has been shown that α_{IIb}β₃ and αᵥβ₃ posses endogenous thiol isomerase activity (O'Neill S, Robinson A, Deering A, Ryan M, Fitzgerald D J, Moran N. The platelet integrin a_{IIb}b₃ has an endogenous thiol isomerase activity. J. Biol. Chem. 2000; 275:36984-36990), but it is not known if this activity is sufficient to promote the conformational change in either direction. However, there must be an additional level of regulation to prevent the receptor being presented in a constituitively active form. It is possible that this could involve PDI, although, to date, the only functional association on the platelet surface that has been shown for PDI is with glycoprotein 1b+ and not α_{IIb}β₃ (Burgess J K et al, supra)

The present invention is based on the identification and isolation by the inventors of an additional thiol isomerase enzyme from the surface of human platelets. This protein has been identified as ERP5, which is a putative member of the thiol isomerase family and was first identified by Hayano T, et al (Hayano T, Kikuchi M. Cloning and sequencing of the cDNA encoding human P5. Gene. 1995; 164:377-378) the entire contents of which is incorporated herein by reference.

It has further been discovered by the inventors from studies of platelet membrane fractions from resting platelets that the majority of ERP5 present on the membranes of platelets is present on internal membranes. However, the internal pool of ERP5 can be mobilised rapidly in response to stimulation with physiological agonists.

This discovery is surprising since proteins that possess a KDEL retention sequence are usually only found associated with internal membranes such as the endoplasmic reticulum. The inventors are the first to identify the presence of ERP5 on platelet membranes, and that upon exposure of the platelets to a platelet agonist ERP5 is rapidly recruited to the external platelet membranes. Antibodies generated by the inventors that inhibit the thiol isomerase activity of ERP5 can block/reduce platelet activation and aggregation in response to platelet agonists.

The inventors are also the first to show that human ERP5 possesses the predicted thiol isomerase activity.

### SUMMARY OF THE INVENTION

According to a first aspect of the present invention there is provided a method of treating a disease or condition mediated by platelet activation comprising supplying a therapeutically effective amount of an ERP5 inhibitor to a patient in need thereof, so as to reduce the effects of platelet activation.

In the context of the present invention an inhibitor of ERP5 is a compound that is capable of specifically binding to ERP5 and thereby inhibiting platelet activation in response to at least one platelet agonist.

Inhibitors include any compound or molecule that inhibits the binding of the ERP5 to its platelet associated receptor integrin β₃ or alternatively inhibits the expression or thiol isomerase activity of the ERP5 itself. For example, in this context the inhibitor may either be an antibody specific for ERP5 itself or alternatively a small molecule, a protein, peptide, small peptide or oligopeptide, nucleic acid or oligonucleotide or other compound which inhibits ERP5 on platelet membranes.

By small molecule it is generally meant herein a molecular entity with a molecular weight of less than 1500, preferably less than 1000. This may for example be an organic, inorganic or organometallic molecule, which may also be in the form or a suitable salt, such as a water-soluble salt; and may also be a complex, chelate and/or a similar molecular entities, as long as its (overall) molecular weight is within the range indicated above.

The term “small peptide” generally covers (oligo) peptides that contain a total of between 2 and 35, such as for example between 3 and 25, amino acids (e.g. in one or more connected chains, and preferably a single chain). It will be clear that some of these small peptides will also be included in the term small molecule as used herein, depending on their molecular weight.

It will be understood that suitable inhibitors can be identified by means of the screening assay described herein. Compounds identified by the screening assay can be used as inhibitors in the methods of the invention.

In a preferred embodiment, the inhibitor is an anti-ERP5 antibody.

In the context of the present invention a platelet agonist should be construed as referring to any compound which interacts with platelets so as to cause them to become activated, such as but not limited to, collagen, thrombin or convulxin. It will be understood by the skilled person that the term platelet activation refers to the processes such as, for example, adhesion, aggregation, fibrinogen binding and P-selectin expression which occur in response to stimulation of resting platelets by exposure to an external stimulus.

Preferably, the disease to be treated is thrombosis. Thrombosis is the abnormal clotting of blood within a blood vessel which can lead to a reduction in, or blocking of, the flow of blood. This can lead to a number of serious consequences, such as heart attack or stroke, depending upon where the clot is formed, or to pulmonary embolisms if the clot is carried to the lungs.

Furthermore, when referring to the use of ERP5 inhibitors in the treatment of disease, as would be appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

An antibody for use in the first aspect may be raised according to standard techniques well known to those skilled in the art by using ERP5 protein or a fragment or single epitope thereof as the challenging antigen, an exemplary, non limiting, method of producing the antibody using recombinant ERP5 to raise anti-human ERP5 in rabbits is described herein below.

Reference to such an “antibody” as described above includes not only complete antibody molecules but fragments thereof. Antibody fragments which contain the idiotype of the molecule can be generated by known techniques, for example, such fragments include but are not limited to the F(ab′)₂ fragment which can be produced by pepsin digestion of the antibody molecule; the Fab′ fragments which can be generated by reducing the disulfide bridges of the F(ab′)₂ fragments and the Fab fragments which can be generated by treating the antibody molecule with papain and a reducing agent. Chimeric humanized and fully humanized mAb can now be made by recombinant engineering. By addition of the human constant chain to F(ab′)₂ fragments it is also possible to create a humanized monoclonal antibody which is useful in immunotherapy applications where patients making antibodies against the mouse Ig would otherwise be at a disadvantage. Breedveld F. C. Therapeutic Monoclonal Antibodies. Lancet 2000 Feb. 26; 335, P735-40.

Human platelet activation assays in the present application have been undertaken in vitro, and the effects of anti-ERP5 antibody measured using flow cytometry, as described herein. The skilled person would understand that the results from these assays would be equally applicable to an in vivo environment and that the same or a functionally equivalent anti ERP5 antibody would have utility in methods of treatment.

According to a second aspect of the present invention there is provided a anti-human ERP5 antibody which specifically binds to cell surface ERP5 antigen so as to inhibit the effects of platelet activation in response to exposure to a platelet agonist. Preferably, said effects of platelet activation which are inhibited by binding of the antibody are platelet aggregation, fibrinogen binding, and surface expression of P-Selectin.

According to a third aspect of the present invention there is provided a pharmaceutical composition comprising an anti-ERP5 antibody and at least one pharmaceutically acceptable diluent or excipient.

It will be understood that the pharmaceutical composition may be administered by any suitable means, such as, but not limited to oral or nasal administration, suppository, subcutaneous injection or intravenous administration.

In the pharmaceutical composition of the invention, preferred compositions include pharmaceutically acceptable carriers including, for example, non-toxic salts, sterile water or the like. A suitable buffer may also be present allowing the compositions to be lyophilized and stored in sterile conditions prior to reconstitution by the addition of sterile water for subsequent administration. The carrier can also contain other pharmaceutically acceptable excipients for modifying other conditions such as pH, osmolarity, viscosity, sterility, lipophilicity, osmobility or the like. Pharmaceutical compositions which permit sustained or delayed release following administration may also be used.

According to a fourth aspect of the present invention, there is provided a method of modulating platelet activation comprising contacting an effective amount of an ERP5 inhibitor with a population of platelets so as to modulate activation in response to platelet agonists. Preferably, the ERP5 inhibitor is an anti-ERP5 antibody.

Preferably, the response to platelet activation which is modulated is platelet aggregation. More preferably, the modulation results in a reduction in platelet aggregation leading to a reduced risk of thrombosis.

Alternatively, the modulation effect may be a reduction in fibrinogen binding activity.

The inventors of the present invention have also discovered that ERP5 becomes associated with the β₃ subunit of the integrin α_{IIb}β₃ in platelets activated by agonists.

According to a further aspect of the present invention there is provided a method for identifying a molecule capable of inhibiting ERP5 modulated activation of platelets comprising the following steps:
a) exposing ERP5 to the molecule of interest,
b) determining the thiol isomerase activity of the ERP5,
c) exposing platelets to those molecules which inhibit ERP5 thiol isomerase activity,
d) activating said platelets by exposure to a platelet agonist,
e) determining the level of platelet aggregation in response to platelet activation,
f) comparing the level of aggregation with platelets not exposed to the molecule of interest,
wherein molecules which reduce the level of aggregation of platelets when compared to a control experiment in which the platelets are not exposed to the molecule are identified as inhibitors of ERP5 modulated platelet activation.

Compounds which are identified are suitable for use in the methods of the current invention along with derivatives that retain substantially the same activity as the starting material, or more preferably exhibit improved activity, which may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutical active agents such as, for example, improved solubility, reduced toxicity, enhanced uptake, etc.

In a preferred embodiment, the thiol isomerase activity is determined by the ability of ERP5 to renature RNAse and aggregation is determined using flow cytometry.

Preferably, the platelets are activated by exposure to collagen, convulxin or thrombin.

### BRIEF DESCRIPTION OF THE DRAWINGS

The invention will be further understood with reference to the following experimental Examples and the accompanying figures in which:

FIG. 1 shows the localisation of ERP5 to internal and external membranes of human platelets. Human platelet internal (40 μg) and external (80 μg) membranes from resting platelets prepared by high-voltage free flow electrophoresis were separated by SDS-PAGE and immunoblotted. ERP5 protein was detected using specific polyclonal antibodies produced by the inventors.

FIG. 2 shows that ERP5 cell surface exposure increases in response to platelet stimulation. Stimulation of platelets by convulxin (A), collagen (B), or thrombin (C) results in a increase in cell-surface expression for ERP5 in a concentration- and time-dependent manner.

i. Concentration-dependence;
A. Basal (shaded), Cvx 10 ng/ml (−), Cvx 40 ng/ml ( . . . );
B. Basal (shaded), Coll 25 μg/ml (−), Coll 100 μg/ml ( . . . )
C. Basal (shaded), Thr 0.2 U/ml (−), Thr 1.0 U/ml ( . . . ) all for 90 s stimulation.

ii. Time dependence;
A. Basal (shaded), t=45 s (−), t=300 s ( . . . ), Cvx 40 ng/ml;
B. Basal (shaded), t=45 s (−), t=300 s ( . . . ), Coll 25 μg/ml;
C. Basal (shaded), t=45 s (−), t=300 s ( . . . ), Thr 1.0 U/ml.

iii. Normalised plots for the increase in cell-surface exposure observed for ERP5 over time are given for convulxin (A), collagen (B), and thrombin (C); mean+/−SE (n=3).

FIG. 3 shows the thiol isomerase activity of human ERP5. Thiol isomerase activity was assessed as the ability to refold denatured, scrambled RNAse and enhance the degradation of cyclic 2′,3′-cytidine monophosphate, as followed by UV/vis spectroscopy.

A. Activity was observed for a recombinant ERP5 fusion protein (2.7 μg) and a recombinant PDI fusion protein (1.4 μg) relative to samples containing only cyclic 2′,3′-cytidine monophosphate (blank), or only fusion protein.

B. Anti-ERP5 polyclonal antibodies raised in sheep were able to partially inhibit the thiol isomerase activity of a recombinant ERP5 fusion protein. Pre-immune IgG was found to possess no such inhibitory activity. Data are mean+/−SE from 3 different determinations, * P<0.05 t-test.

FIG. 4 shows that an inhibitory antibody for ERP5 inhibits platelet aggregation.

Platelets (4×10⁸ cells/ml) were incubated with anti-ERP5 IgG or control IgG at the concentrations given for 2.5 minutes prior to addition of agonist.

A. Collagen, 2.5 μg/ml, incubated with: Pre-immune IgG (36 μg/ml); anti-ERP5 antibody (3 μg/ml); anti-ERP5 antibody (36 μg/ml), as indicated.

B. Convulxin, 30 ng/ml, incubated with: Pre-immune IgG (24 μg); anti-ERP5 antibody (12 μg); anti-ERP5 antibody (24 μg), as indicated.

Prior to addition of antibodies platelets were pre-incubated with a saturating concentration of a F(ab) fragment of the IV.3 protein to prevent signalling through the Fc_{γ}RIIa receptor. Control IgG was purified from the pre-immune serum of the animal used to raise antibodies in. Trace shown is representative from that observed for at least three different donors.

FIG. 5 shows that the binding of fibrinogen is inhibited in platelets following blocking of cell-surface ERP5.

Binding of FITC labelled fibrinogen was measured using flow cytometry on platelets stimulated with the agonists convulxin or collagen. Prior to stimulation platelets were incubated with anti-ERP5 or anti-PDI antibodies or control antibodies from pre-immune sera.

A. Histogram for fluorescence of FITC-fibrinogen labelled platelets in response to the agonist convulxin (100 ng/ml); control IgG (12 μg) (shaded), anti-ERP5 antibody (12 μg) (−), anti-PDI antibody (33 μg) ( . . . )

B. Residual binding of FITC-fibrinogen following incubation of platelets with control IgG (12 μg), anti-ERP5 antibody (12 μg), anti-PDI antibody (33 μg). Agonists used at concentrations of:—convulxin 100 ng/ml; collagen 10 μg/ml. Data are presented as mean+/−S.E for three separate experiments, * P<0.05, ** P<0.005.

FIG. 6 shows that P-Selectin exposure is inhibited in platelets following blocking of cell-surface ERP5.

Binding of PE conjugated anti-CD62p was measured using flow cytometry on platelets stimulated with the agonists convulxin or collagen. Prior to stimulation platelets were incubated with anti-ERP5 or anti-PDI antibodies or control antibodies from pre-immune sera.

A. Histogram for fluorescence of PE anti-CD62p labelled platelets in response to the agonist convulxin (100 ng/ml); control IgG (12 μg/ml) (shaded), anti-ERP5 antibody (12 μg) (−), anti-PDI antibody (33 μg) ( . . . )

B. Residual binding of PE anti-CD62p following incubation of platelets with control IgG (12 μg), anti-ERP5 antibody (12 μg), anti-PDI antibody (33 μg). Agonists used at concentrations of:—convulxin 100 ng/ml; collagen 10 μg/ml

Data are presented as mean+/−S.E for three separate experiments, * P<0.05, ** P<0.005

FIG. 7 shows the stimulation dependent association of ERP5 with integrin β₃

Platelets (1×109 cells/ml) were stimulated in the presence of EGTA, apyrase and indomethacin at varying concentrations of convulxin (ng/ml) or thrombin (U/ml) for 90 s (A), or at fixed concentrations of agonist (Cvx 100 ng/ml, Thr 1.0 U/ml) for increasing duration (B). Following sample lysis proteins were precipitated and separated with specific antibodies and protein A sepharose. Immunoblotting was used to show interacting proteins. Blots were stripped and re-probed to verify equivalent levels of target antigen in each sample lane.

### DETAILED DESCRIPTION OF THE INVENTION

Platelet agonists were; collagen (Horm, type I from equine tendon, Nycomed), convulxin, and thrombin (Sigma). Platelet membranes were purified by free flow electrophoresis. Horseradish peroxidase-conjugated secondary antibodies and the enhanced chemiluminescence detection system from Amersham Biosciences; RNAse (Roche); bovine serum albumin (First Link); monoclonal anti-PDI, MA3-019 (Affinity Bioreagents); PE-conjugated anti-CD62p, P-Selectin, (PharMingen). All other reagents were purchased from Sigma (Poole, UK).

Protein concentrations were determined from a Bradford assay using the BioRad protein assay kit with a bovine gamma-globulin standard.

Antibody Generation

The full-length ERP5 gene (cDNA clone provided by Prof. M. Kikuchi Ritsumeikan University, Japan) was cloned into the pGEX4T2 prokaryotic expression vector and recombinant protein purified from E. coli. Polyclonal antibodies were raised against the fusion protein and further purified against protein G-sepharose or recombinant ERP5 linked sepharose affinity columns. Specificity was determined by immunoblotting platelet lysates with the antibodies and comparing with antibody neutralised using recombinant ERP5, and anti-PDI and anti-CaBP1 antibodies.

Preparation and Stimulation of Washed Platelets

Human platelets from drug-free volunteers were prepared on the day of the experiment by differential centrifugation as described previously (Gibbins J, Asselin J, Farndale R, Barnes M, Law C-L, Watson S P. Tyrosine phosphorylation of the Fc receptor g-chain in collagen-stimulated platelets. J. Biol. Chem. 1996; 271:18095-18099) and suspended in modified Tyrode's/Hepes buffer (134 mM NaCl, 0.34 mM Na₂HPO₄, 2.9 mM KCl, 12 mM NaHCO₃, 20 mM Hepes, 5 mM glucose, 1 mM MgCl₂, pH 7.3). Polyclonal anti-PDI antibodies were raised in rabbits using purified recombinant, His-tagged, PDI.

Stimulation of platelets with collagen (Coll), convulxin (Cvx), and thrombin (Thr) was performed in an optical aggregometer (Chrono-log) at 37° C. with continuous stirring. For platelet aggregation and flow cytometry studies platelets were stimulated at a concentration of 4×10⁸ cells/ml, for immunoprecipitation studies platelets were stimulated at a concentration of 1×10⁹ cells/ml. Where necessary non-aggregating conditions were maintained by the addition of EGTA (1 mM), and secondary stimulation by released thromboxane A₂ or secreted ADP was prevented by inclusion of indomethacin (10 μM), and apyrase (2 U/ml).

Co-Immunoprecipitation

Standard procedures for immunoprecipitation were followed (Cicmil M, Thomas J M, Sage T, et al. Collagen, convulxin, and thrombin stimulate aggregation-independent tyrosine phosphorylation of CD31 in platelets: Evidence for the involvement of Src family kinases. J. Biol. Chem. 2000; 275:27339-27347). Following stimulation platelets were lysed with ice-cold NP-40 buffer (300 mM NaCl, 20 mM Tris, 10 mM EDTA, 2% NP-40, 1 mM phenylmethylsulfonyl fluoride, 2 mM Na₃VO₄, 10 g/ml leupeptin, 10 μg/ml aprotonin, and 1 μg/ml pepstatin A, pH 7.3). Detergent insoluble debris was removed, samples pre-cleared with protein A (or G) sepharose, and then incubated with specific antibodies and protein A (or G) sepharose at 4° C. with rotation for 90 minutes. Following Western blotting, PVDF membranes were blocked by incubation in 5% (w/v) bovine serum albumin. Primary and secondary antibodies were diluted in tris-buffered saline/Tween (TBS/T; 20 mM Tris, 137 mM NaCl, 0.1% (v/v) Tween 20, pH 7.6) containing 2% (w/v) bovine serum albumin and incubated with PVDF membranes for 90 mins at room temperature. Blots were washed for 90 mins in TBS/T following incubation and then developed using the enhanced chemiluminescence detection system. Primary antibodies were used at a concentration of 1 μg/ml. Horseradish peroxidase conjugated secondary antibodies were diluted 1:10,000. Western blots were stripped in a low pH buffer (0.1M acetate pH 4, 0.5M NaCl) for 30 mins and then re-blocked by incubation with 5% (w/v) bovine serum albumin prior to re-probing to test for the presence of the original antigen. ECL images were collected on X-ray film. Densitometry was employed to verify equal loading using a BioRad GS710 densitometer and Quantity One software package.

Flow Cytometry

Human platelets were stimulated at a density of 4×10⁸ cells/ml with the appropriate agonist in the presence of EGTA (1 mM), indomethacin (10 μM), and apyrase (2 U/ml). Stimulation was terminated by dilution to 2×10⁸ cells/ml by the addition of modified tyrodes buffer (Tyrode's/Hepes, pH 7.3 containing 1% (w/v) bovine serum albumin, 1 mM EGTA, 200 μM sodium azide). Primary antibody was added at appropriate dilutions and incubated for 1 hr on ice. Secondary antibody (fluorescein isothiocyante-conjugated IgG) was used at 1:2000 dilution and incubated for 1 hr on ice in the dark. Data were collected and analysed using a Becton Dickinson FACScan flow cytometer and CELLQuest software.

Fibrinogen Binding

The assay was employed as given above with the omission of EGTA from all buffers and using FITC labelled human fibrinogen. To examine the effect of antibody blocking samples were incubated with α-ERP5 antibodies, or control antibodies from pre-immune serum, prior to platelet stimulation. All samples were pre-incubated with saturating concentrations of a F(ab) fragment of the protein IV.3 to prevent signalling through the Fc_{γ}RIIA receptor

RNAse Activity Assay

Thiol isomerase activity was assessed by the ability to renature RNAse that had been inactivated by reduction and denaturation (rdRNAse). The assay was performed as outlined by Pigiet et al with minor modifications (O'Neill S et al, supra, Pigiet V P, Schuster B J. Thioredoxin-catalyzed refolding of disulfide containing proteins. Proc. Nat'l Acad. Sci. USA. 1986; 83:7643-7647). Reactivated RNAse was assayed by the degradation of cyclic 2′-3′ cytidine monophosphate (cCytP), measured by the increase in absorbance at 284 nm. Controls of rdRNAse only, cCytP substrate only (blank), protein and cCytP substrate but no rdRNAse, were run with each set of experiments to test for RNAse contamination in samples or buffers. The activity was expressed relative to native RNAse, or as a percentage of inhibition of activity for antibody blocking experiments.

Data Analysis

Data was analysed using the SPSS software package and sample correlation determined using a two-tailed paired t-test at a 95% confidence value.

### EXAMPLES

### EXAMPLE 1

### ERP5 is Present in Human Platelets—Associated with Internal and External Membranes

An unknown protein was reproducibly isolated from human platelet membrane fractions using a convulxin affinity column. Convulxin is a protein component from the venom of the rattlesnake that possesses a high affinity for the platelet glycoproteins GP VI and GP 1b (Polgár J, Clemetson J M, Kehrel B E, et al. Platelet Activation and Signal Transduction by Convulxin, a C-type Lectin from Crotalus durissus terrificus (Tropical Rattlesnake) Venom via the p62/GPVI Collagen Receptor. J. Biol. Chem. 1997; 272:13576-13583, Kanaji S, Kanaji T, Furihata K, Kato K, Ware J L, Kunicki T J. Convulxin Binds to Native, Human Glycoprotein Ib. J. Biol. Chem. 2003; 278:39452-39460). N-terminal sequence data was obtained for the first fifteen residues of the protein (LYSSSDDVIELTPSN). A BLAST search revealed this to be identical to the sequence of ERP5 following cleavage of a predicted signal sequence, allowing the protein to be identified.

ERP5 was cloned by Hayano et al from a placental cDNA library while looking for proteins related to the thiol isomerase enzyme protein disulphide isomerase, PDI (Hayano T, Kikuchi M. Cloning and sequencing of the cDNA encoding human P5. Gene. 1995; 164:377-378). The gene sequence encodes a 48 kDa protein containing; two active thioredoxin domains (—CGHC—) that share 47% aa sequence identity with human PDI, a C-terminal peptide binding domain, and a KDEL sequence for retention in the endoplasmic reticulum (Hayano T, Kikuchi M.supra). Sequence alignment studies by Ferrari and Soling suggest that ERP5 and PDI share a similar domain structure, but with ERP5 containing one less thioredoxin-like domain (Ferrari D M et al, supra, Kramer B, Ferrari D M, Klappa P, Pohlmann N, Soling H-D. Functional roles and efficiencies of the thioredoxin boxes of calcium-binding proteins 1 and 2 in protein folding. Biochem. J. 2001; 357:83-95).

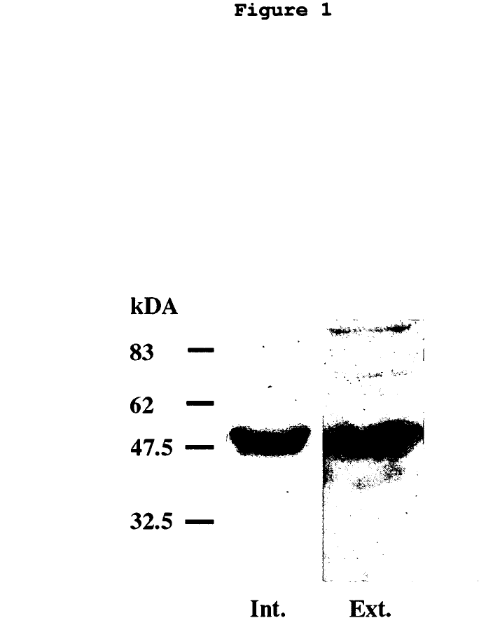

Given that this protein is normally restricted to the ER the presence of ERP5 in membrane fractions was confirmed by immunoblot analysis (not shown). The membrane association was examined more closely using internal and external membranes from resting platelets isolated by free-flow electrophoresis (provided by Dr K Authi), FIG. 1. These showed that ERP5 was present on both internal and external membranes. The higher loading and longer exposure times required to observe the protein from external membranes indicates there are relatively lower levels of ERP5 present on the external plasma membranes of resting platelets. ERP5 was detected using polyclonal antibodies raised in rabbits and sheep. The antigen used was a GST-fusion protein of the full-length human ERP5. These antibodies recognised a protein on Western blots of the same mobility as that recognised by antibodies to CaBP1, the rat homologue of ERP5 (Kramer B et al, supra).

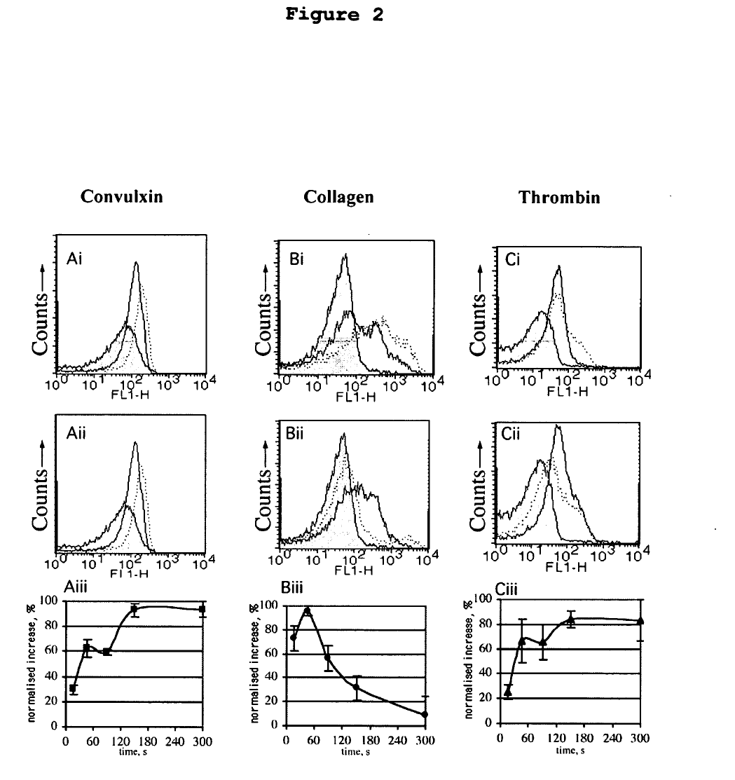

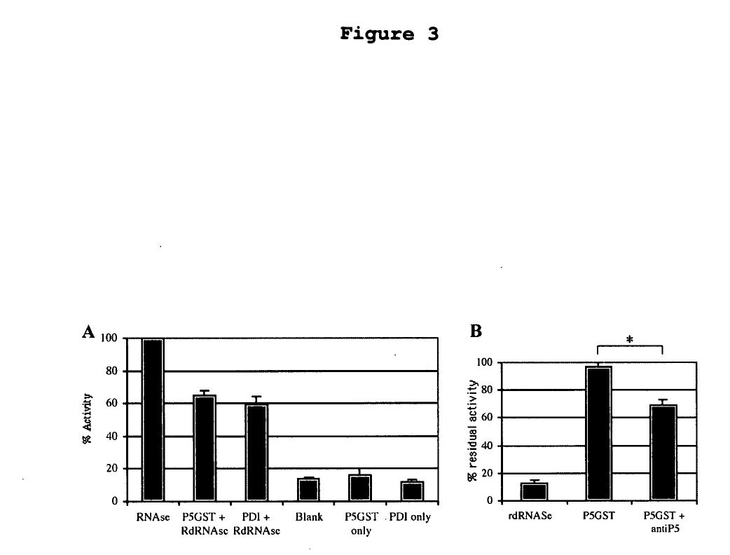

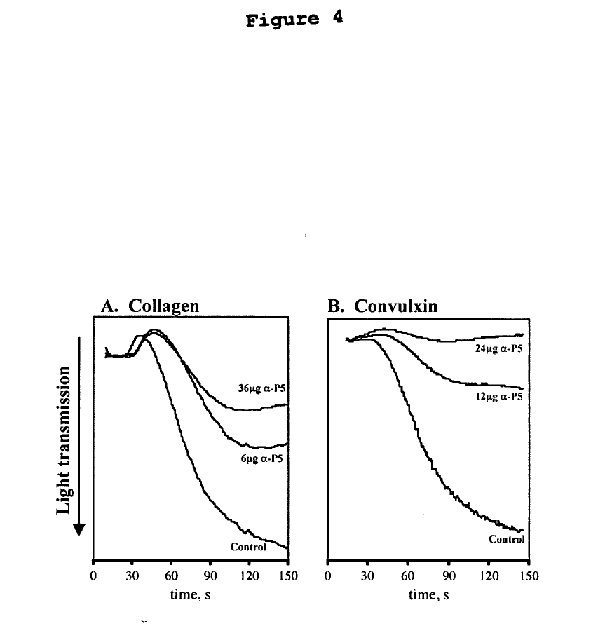

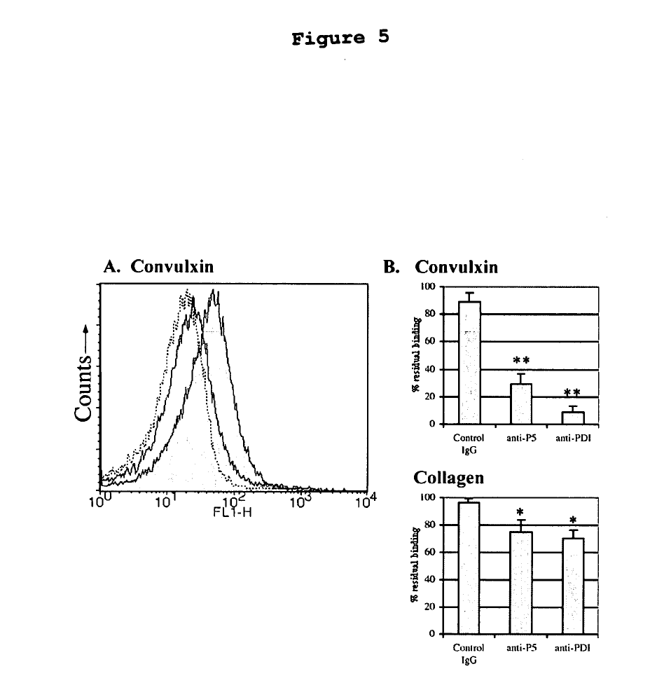

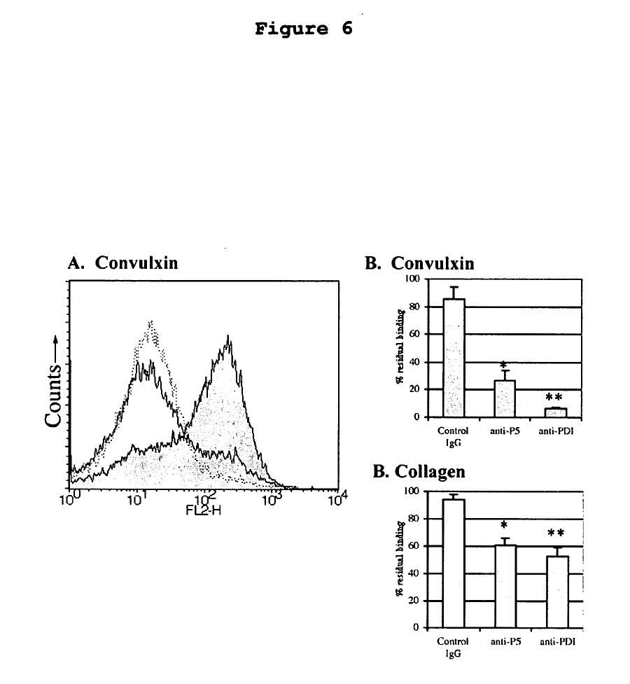

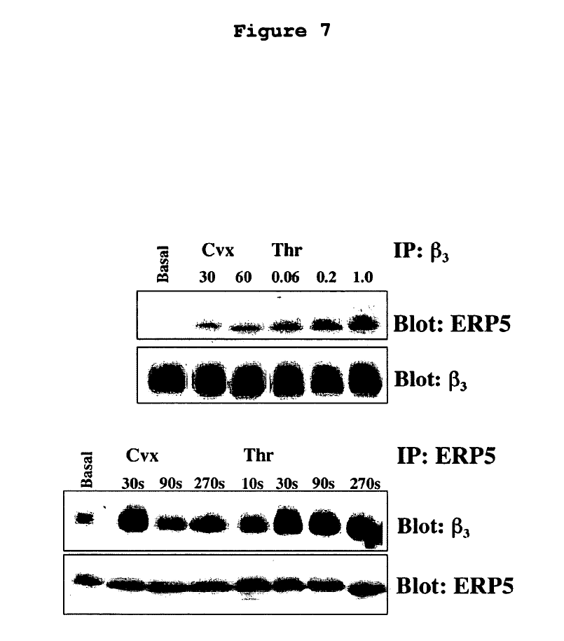

Flow cytometry was employed to confirm cell surface expression of ERP5 and investigate whether this was a static or dynamic process. Washed platelets were stimulated with the agonists convulxin, collagen or thrombin and the concentration- and time-dependent patterns of cell-surface exposure for ERP5 were studied, FIG. 2. Low levels of cell-surface ERP5 were found to be substantially and rapidly increased following stimulation with each agonist in a concentration-dependent manner. To investigate the trends observed in the time-dependence for the increase in cell-surface exposure for ERP5 the data was normalised to the greatest response for an individual experiment, and averaged to overcome donor variability (FIG. 2.iii). All three agonists demonstrated biphasic profiles, where there was an initial rapid increase in cell-surface exposure, which peaks at approximately 45 s, with substantial increases seen as rapidly as 15 s. For convulxin and thrombin this was followed by a lag phase where exposure levels were maintained or dipped slightly between 60 s and 120 s before beginning to rise again over the period of 150-300 s. The data suggest that there are secondary effectors or mechanisms present for promoting a second wave of cell-surface exposure of ERP5 in response to the agonists. The experiments were performed in the presence of EGTA, apyrase, and indomethacin which block the second wave of platelet aggregation responses based on fibrinogen, ADP, and thromboxane A2. Microaggregates based upon α_{IIb}β₃ interactions have been reported to form in the presence of EGTA (Jones K L, Hughan S C, Dopheide S M, Farndale R W, Jackson S P, Jackson D E. Platelet endothelial cell adhesion molecule-1 is a negative regulator of platelet-collagen interactions. Blood. 2001; 98:1456-1463) and this may have accounted for the second wave of signalling. A different profile was observed for the time-dependent increase in cell-surface exposure of ERP5 with the agonist collagen. Again there was a rapid increase which peaked at approximately 45 s but subsequently these levels decreased to approximately baseline values by 300 s.

### Example 2

### ERP5 Protein has Thiol Isomerase Activity

Based upon the refolding of reduced denatured RNASe previous studies have demonstrated thiol isomerase activity for bovine liver ERP5, CaBP1, the rat homologue to ERP5, and human PDI (Kramer B et al, supra). To verify that human ERP5 is a functionally active thiol isomerase we analysed the recombinant fusion protein in this assay system (FIG. 3). The protein was found to possess thiol isomerase activity, with activity approximately 70% of that measured for molar equivalents of a PDI recombinant fusion protein. The difference in activity observed may have been due to constraints from the fusion partners of these proteins or based upon inherent differences in activity (as observed for PDI and CaBP1 (Kramer B et al, supra)). Human ERP5 immunoprecipitated from platelet samples using a non-blocking antibody also demonstrated thiol isomerase activity (data not shown). It was found that under the assay conditions employed the thiol isomerase activity of both ERP5GST and PDIHis was dependent on divalent cations and inhibited in the presence of EDTA (not shown). This was opposite to the observed thiol isomerase activity profile for the integrin sub-unit 3, which has been shown to display enhanced activity in the presence of EDTA (O'Neill S. et al, supra). Such differential cation-dependence for thiol isomerase activity may be important in the cellular regulation of the activity of these proteins.

Antibodies that block the activity of PDI have been reported. The effect on enzymatic activity of ERP5 by antibodies to ERP5 was investigated. Antibodies raised in sheep against recombinant ERP5 were found to inhibit enzyme activity, where pre-immune IgG displayed no activity (FIG. 3B). It was not possible to completely block the thiol isomerase activity of ERP5GST, even at very high antibody concentrations, which is consistent with studies performed on PDI with blocking antibodies.

### Example 3

### Platelet Aggregation is Inhibited by Antibody Blocking of ERP5

Activity-blocking anti-ERP5 antibodies were used to investigate the potential involvement of ERP5 in the regulation of platelet function. Platelets were stimulated with collagen or convulxin following incubation with anti-ERP5 antibodies or control IgG purified from the pre-immune serum of the animal used to raise the antibodies. Prior to addition of inhibitory antibodies platelets were incubated with saturating concentrations of the F(ab) fragment of the IV.3 protein to prevent signalling through the Fc_{γ}RIIa receptor (Cicmil M et al, supra). The traces shown in FIG. 4 demonstrate that anti-ERP5 antibodies are capable of blocking the aggregation response induced by low concentrations of convulxin and collagen. In response to low concentrations of collagen platelet aggregation was substantially reduced by 6 μg/ml anti-ERP5. The aggregation profile was reversible with platelets showing signs of dis-aggregating after 120 s. Addition of higher concentrations of anti-ERP5 antibodies further decreased the level of aggregation observed, although it was not possible to fully inhibit aggregation responses. For convulxin, aggregation was substantially reduced following incubation with 12 μg/ml of antibody and it was possible to completely inhibit aggregation at higher antibody concentrations. For both collagen and convulxin pre-incubation with antibodies did not inhibit shape change. At higher concentrations of collagen and convulxin it was possible to overcome the inhibitory effects of anti-ERP5 antibodies.

### Example 4

### ERP5 is Involved in the Regulation of Fibrinogen Binding

Investigations into the ability of platelets to bind fibrinogen in the presence and absence of inhibitory anti-ERP5 antibodies were undertaken. Flow cytometry was used to measure the binding of FITC-labelled fibrinogen to collagen and convulxin stimulated platelets (FIG. 5). Stimulation of platelets resulted in an increase in the level of binding of FITC-fibrinogen, consistent with an increase in its affinity to integrin α_{IIb}β₃ (Shattil S J, Kashiwagi H, Pampori N. Integrin Signaling: The Platelet Paradigm. Blood. 1998; 91:2645-2657, D. R. Phillips, I. F. Charo, R. M. Scarborough. GPIIb-IIIa: The responsive integrin. Cell. 1991:359-362). Incubation of platelets with either anti-ERP5 antibodies or monoclonal anti-PDI antibodies resulted in a significant decrease in platelet binding to fibrinogen for both agonists. In response to convulxin stimulation platelet binding of fibrinogen was reduced by 70% and 91% for anti-ERP5 (P<0.005) and anti-PDI (P<0.005) antibodies respectively. When collagen was used as an agonist the reduction in fibrinogen binding was more modest, at 25% and 29% for anti-ERP5 (P<0.05) and anti-PDI (P<0.05) antibodies respectively. This is consistent with the more modest inhibitory effect of anti-ERP5 on platelet aggregation. Pre-incubation of platelets with IgG from pre-immune sera had no effect on levels of FITC-fibrinogen binding.

### Example 5

### α-Granule Secretion is Inhibited by Anti-ERP5

The cell-surface exposure of P-selectin, a membrane component of α-granules, is used commonly as a marker for the state of platelet activation and degranulation (Palabrica T, Lobb R, Furie B C, et al. Leukocyte accumulation promoting fibrin deposition is mediated in vivo by P-selectin on adherent platelets. Nature. 1992; 359:848-851, Furie B, Furie BC, Flaumenhaft R. A journey with platelet P-selectin: the molecular basis of granule secretion, signalling and cell adhesion. Thromb. Haemost. 2001; 86:214-221). Surface expression of P-selectin concomitant with binding of FITC-fibrinogen was measured using flow cytometry (FIG. 6). P-selectin exposure was inhibited significantly in response to the agonists collagen and convulxin by blocking ERP5 or PDI with specific function-blocking antibodies. In response to convulxin stimulation there was a decrease in surface expression for P-Selectin of 73% and 94% in the presence of anti-ERP5 (P<0.01) and anti-PDI (P<0.001) antibodies respectively, while in response to collagen there was a decrease of 39% (P<0.02) and 46% (P<0.005). Analysis of the fluorescence profile for P-selectin expression following convulxin stimulation indicated that there were two main populations of platelets, showing low or high levels of fluorescence. The profile for fibrinogen binding showed a more normal distribution with a progressive increase in fluorescence with increasing concentrations of agonist. Scatter graph analysis of FITC-fibrinogen fluorescence relative to phycoerythrin conjugated P-selectin fluorescence demonstrated that cells lacking surface P-selectin exposure also bound fibrinogen poorly (data not shown).

The data obtained in the thiol isomerase assay highlights that complete inhibition of activity was not achieved using blocking antibodies against recombinant ERP5 or recombinant PDI. Thus, it is not possible to fully inhibit the activity of one protein using inhibitory antibodies. Therefore, one can not directly relate the levels of inhibition seen in the fibrinogen binding and P-Selectin expression assays to the relative contributions of the ERP5 and PDI proteins to these processes. However, these data strongly implicate cell-surface ERP5 and PDI to be involved in the regulation of platelet thrombus formation.

### Example 6

### ERP5 Associates with Integrin 3 in Stimulated Platelets

The flow cytometry data for fibrinogen binding demonstrated that blocking ERP5 with specific antibodies inhibited fibrinogen binding by stimulated platelets (FIG. 5). To investigate whether there was a direct association between ERP5 and the integrin responsible for fibrinogen binding, α_{IIb}β₃, co-immunoprecipitation studies were performed. It was found that the integrin 3 sub-unit became associated with ERP5 in platelets activated by the agonists convulxin and thrombin (FIG. 7). This association was observed using complimentary experimental techniques (i.e. for immunoprecipitation using either anti-ERP5 or anti-β₃ antibodies). The degree of association was both agonist concentration and time dependent; increasing with increasing concentrations of agonist; and peaking at approximately 30 s post-stimulation. The data shown in FIG. 2.iii, for the cell-surface exposure of ERP5, demonstrate that there is a peak at approximately 45 s for the exposure of ERP5 in response to both of the agonists convulxin and thrombin.

Similar experiments were performed to examine the potential interaction of PDI with 3, but such an interaction was not observed. Nor was any interaction between ERP5 and PDI observed.

A number of recent studies have developed the concept of redox controlled receptor remodelling as part of the activation process in platelets. It has been proposed that these reactions are based upon thiol isomerase activity, the ability to generate, reduce, or rearrange disulphide bonds in proteins (Yan B, Smith J W. supra, Jiang X-M, et al, supra, O'Neill S et al, supra). Resting platelets display low levels of thiol isomerase activity on the cell surface, and these levels are dramatically enhanced when platelets are stimulated by agonists (Burgess J K et al, supra). The functional importance of this activity is demonstrated by the fact that blocking thiol isomerases, inhibits a number of key events in the platelet activation process including adhesion, aggregation, fibrinogen binding and P-Selectin expression (Essex D W, Li M, supra, Lahav J, Jurk K, et al, supra, Lahav J, Wijnen E M, et al, supra). The only thiol isomerase enzyme characterised in platelets previously has been protein disulphide isomerase. The present application demonstrates the presence of an additional thiol isomerase enzyme, ERP5, on the surface of platelets. The contribution to the cell-surface thiol isomerase activity by enzymes, such as ERP5 could be the basis for the observation that chemical modification reagents consistently inhibit platelet activation markers to a greater extent than specific antibodies that inhibit PDI.

In theory a small number of thiol isomerases could activate a large number of receptors as they do not have to form long-term stable complexes with their substrates. The balance in this scenario will be time because fewer proteins will take longer to activate all receptors. Indeed, limiting surface expression could be seen as another form of setting the gain, or threshold, for platelet activation by modulating the response time for complete activation. This characteristic of extended periods of shape change and slower onset of aggregation is observed when platelets are incubated with low levels of inhibitory antibodies.

Shuttling of receptors between internal organelles and the cell-surface is a common phenomenem and recent studies have shown that cell-surface expression of GluR5 kainate receptors is regulated by an endoplasmic reticulum retention signal (Ren Z, Riley N J, Needleman L A, Sanders J M, Swanson G T, Marshall J. Cell surface expression of GluR5 Kainate receptors is regulated by an endoplasmic reticulum retention signal. J. Biol. Chem. 2003; 278:52700-52709). Both of the thiol isomerases ERP5 and PDI are recruited to the cell surface in response to stimuli in a concentration and time dependent manner, as shown in FIG. 2 and by Burgess et al supra respectively. The time dependence for cell-surface exposure of ERP5 demonstrates different profiles; for the agonists convulxin and thrombin there is a biphasic profile with an initial peak at approximately 60 s followed by a prolonged increase in exposure. For collagen, following an initial peak in exposure, cell surface levels of ERP5 return to basal after 5 mins. It may be that stimulation with collagen is unable to mobilise a second wave of cell-surface exposure for ERP5, or that receptor-enzyme internalisation is occurring.

Until recently it would have been easy to attribute the differences observed in the time-dependent expression profiles to the fact that all three agonists stimulate platelets through different signalling pathways. Thrombin through the G-protein-coupled-receptor pathway via PAR1 and PAR4 (Vu T K, Hung D T, Wheaton V I, Coughlin S R. Molecular cloning of a functional thrombin receptor reveals a novel proteolytic mechanism of receptor activation. Cell. 1991; 64:1057-1068, Kahn M L, Zheng Y W, Huang W, et al. A dual thrombin receptor system for platelet activation. Nature. 1998; 394:690-694), collagen through the integrin α₂β₁ (Morton L F, Hargreaves P G, Farndale R W, Young R D, Barnes M J. Integrin α₂β₁-independent activation of platelets by simple collagen-like peptides: collagen tertiary (triple-helical) and quaternary (polymeric) structures are sufficient alone for alpha 2 beta1-independent platelet reactivity. Biochem. J. 1995; 306:337-344), and convulxin through the receptor GPVI (Polgár J, et al, supra) alone. However, recent reports have suggested that there is not such a clear distinction; convulxin has been shown to bind GP1b (Kanaji S, et al, supra); and it has been proposed that the central receptor responsible for collagen signalling is GPVI (Nieswandt B, Watson S P. Platelet-collagen interaction: is GP VI the central receptor? Blood. 2003; 102:449-461), with integrin α₂β₁ being responsible primarily for adhesion. Thus, one may expect thrombin to be distinct and not collagen. Previous reports have indicated a link between thiol isomerase activity of PDI and integrin activation. It is possible, therefore, that the different profiles seen are based upon a separate pathway following activation via integrins as opposed to other stimuli. In platelets Wang et al have observed internalisation of soluble collagen via the integrin α₂β₁ over a period of 30 minutes (Wang Z, Leisner T M, Parise L V. Platelet α₂β₁ integrin activation: contribution of ligand internalization and the 2-cytoplasmic domain. Blood. 2003; 102:1307-1315). This may suggest that the profile observed for cell-surface exposure of ERP5 is effected by enzyme re-internalisation in response to stimulation with this agoinst.

The observation that small, thiol-reactive, reagents are effective for thiol isomerase inhibition studies indicates that the enzymatic activity of the proteins underlies their function on the cell-surface (Essex D W, Li M, et al, supra). Little is known, however, of the mechanism by which this occurs. Essex et al have proposed a mechanism for PDI activity whereby it acts downstream of the primary activation process, but prior to the activation of the integrin receptor α_{IIb}β₃ (Essex D W, Li M, et al, supra). They observed that anti-PDI inhibitory antibodies were able to block conversion of integrin α_{IIb}β₃ to the activated state recognised by the PAC-1 antibody, but not block activation via a peptide (LSARLAF) that has been shown to bind α_{IIb}β₃ and directly stimulate aggregation and secretion. On the other hand, reagents that react with thiol groups were able to block activation of α_{IIb}β₃ even when stimulated with the peptide.

The similarity in effects observed for blocking ERP5 and PDI suggests that the two proteins may be acting through a common pathway. This is supported by the widespread ability of this class of proteins to refold scrambled denatured RNAse in the course of the thiol isomerase activity assay. However, it is important to remember the distinction between interacting with a polypeptide chain and with a correctly folded protein on the surface of a cell. Thus, there remains the possibility that although these proteins may share a common pathway and be able to complement one another they may also be able to modulate the activity of different receptors individually.

The data presented in FIGS. 5, 6, and 7; for the binding of fibrinogen, cell-surface exposure of P-Selectin, and co-association of ERP5, highlight the potential inter-relationships between different thiol isomerase enzymes and receptor activation on the platelet cell-surface. Following pre-incubation of platelets with function-blocking antibodies to either ERP5 or PDI the binding of fibrinogen and cell-surface exposure of P-Selectin was found to be significantly inhibited in activated platelets. Differences were observed in agonist and inhibitory antibody responses; with greater inhibition observed for convulxin rather than collagen, and for blocking PDI rather than ERP5. The different levels of inhibition observed may be based upon a difference in potencies of the two agonists used. Previous studies have also investigated the inhibition of fibrinogen binding by activated platelets in response to function-blocking antibodies to PDI. Lahav et al report an approximate 55% decrease in fibrinogen binding and approximate 30% decrease in P-Selectin exposure for collagen stimulated platelets pre-incubated with the monoclonal anti-PDI antibody RL-90 (Lahav J, Jurk K, et al, supra). These results are consistent with the data we have obtained showing a 29% decrease in fibrinogen binding and 47% decrease in P-Selectin exposure in response to blocking with the monoclonal anti-PDI antibody Ma3-019. The greater inhibition observed in this study for PDI relative to ERP5 may be based upon different affinities of the antibodies used, and not reflect the overall enzyme contributions to these events. However, it is interesting to note that the extent of inhibition of fibrinogen binding and P-Selectin exposure is closer for anti-PDI and anti-ERP5 blocking in response to collagen (P>0.2) than in response to convulxin (P<0.05), suggesting that PDI may be more important than ERP5 upon convulxin stimulation. This may be based upon the different signalling pathways affected by these agonists. Convulxin is capable of stimulating both GPVI and GPlb, and Burgess et al have demonstrated that there is a physical association between PDI and GP1b (Burgess J K, et al, supra).

Remodelling of the integrin α_{IIb}β₃ from a blocked to a ligand-binding state involves a conformational change in which the disulphide bonding pattern of the receptor is changed (Shattil S J, et al, supra, D.R. Phillips, et al, supra). The 3 sub-unit of the integrin possess inherent thiol isomerase activity (O'Neill S, et al, supra), although it is not known if this activity is sufficient to promote the conformational change in either direction. We have demonstrated a co-association of ERP5 with the β₃ sub-unit of integrin α_{IIb}β₃ in activated platelets. We propose that when associated with 3 ERP5 is able to assist in the conformational change of the integrin from an active to an inactive state. The mechanism through which this is regulated is uncertain, with increased cell-surface exposure and β3 association likely to be involved. The interaction with, and regulation by, other molecules, such as the interaction of PDI, or calreticulin (Elton C M, Smethurst P A, Eggleton P, Farndale R W. Physical and functional interaction between cell-surface calreticulin and the collagen receptors integrin 2 μl and glycoprotein VI in human platelets. Thromb Haemost. 2002; 88:648-654), may also play a role. Previous studies have been unable to demonstrate an interaction between PDI and β₃ (O'Neill S, et al, supra). No observation of any association between PDI and 3, or ERP5 and PDI was observed in this application.

This application that three main responses can be seen as part of the platelet activation process:—
1. Increased cell-surface presentation of the thiol isomerases PDI and ERP5;
Activation of integrin α_{IIb}β₃ and fibrinogen binding;
3. Increased cell-surface presentation of P-Selectin.

In response to blocking either PDI or ERP5 with specific inhibitory enzymes decreases in fibrinogen binding and P-Selectin expression, responses 2 and 3, have been observed. It is also thought that the addition of blocking agents prior to platelet stimulation also attenuates the increased cell-surface exposure of thiol isomerase enzymes, response 1. In this model platelet stimulation promotes an increase in cell-surface exposure of thiol isomerase enzymes by a pathway common to both ERP5 and PDI. Once on the platelet surface ERP5 and PDI may then act independently to modulate the reactive state of cell-surface receptors, such as β₃.

### REFERENCES

Martire G, Mottola G, Pascale M C, et al. Different fate of a single reported protein containing KDEL or KDXX targeting signals stably expressed in mammalian cells. J. Biol. Chem. 1996; 271:3541-3547
Ferrari D M, Soling H-D. The protein disulphide isomerase family: Unraveling a string of folds. Biochem. J. 1999; 339:1-10
Teasdale R D, Jackson M R. Signal-mediated sorting of membrane proteins between the endoplasmic reticulum and the golgi apparatus. Annu. Rev. Cell Dev. Biol. 1996; 12:27-54
Essex D W, Li M. Protein disulfide isomerase mediates platelet aggregation and secretion. Br. J. Haematology. 1999; 104:448-454
Tager M, Kroning H, Thiel U, Ansorge S. Membrane-bound protein disulphide isomerase (PDI) is involved in regulation of surface expression of thiols and drug sensitivity of B-CLL cells. Experimental Hematology. 1997; 25:601-607
Ryser H J, Levy E M, Mandel R, DiSciullo G J. Inhibition of Human Immunodeficiency Virus Infection by Agents that Interfere with Thiol-Disulfide Interchange Upon Virus-Receptor Interaction. Proc. Nat'l Acad. Sci., USA. 1994; 91:4559-4563
Hotchkiss K A, Matthias L J, Hogg P J. Exposure of the cryptic Arg-Gly-Asp sequence in thrombospondin-1 by protein disulfide isomerase. Biochim. Biophys. Acta. 1998; 1388:478-488
Terada K, Manchikalapudi P, Noiva R, Jauregui H O, Stockert R J, Schilsky M L. Secretion, surface localisation, turnover, and steady state expression of protein disulfide isomerase in rat hepatocytes. J. Biol. Chem. 1995; 270: 20410-20416
Akagi S, Yamamoto A, Yoshimori T, Masaki R, Ogawa R, Tashiro Y. Distribution of protein disulfide isomerase in rat hepatocytes. J. Histochemistry and Cytochemistry. 1988; 36:1533-1542
Kroning H, Kahne T, Ittenson A, Franke A, Ansorge S. Thiol-proteindisulfide-oxidoreductase (protein disulfide isomerase): A new plasma membrane constituent of mature human B lymphocytes. Scand. J. immunol. 1994; 39:346-350
Mandel R, Ryser H J-P, Ghani F, Wu M, Peak D. Inhibition of a reductive function of the plasma membrane by bacitracin and antibodies against protein disulphide isomerase. Proc. Natl. Acad. Sci. USA. 1993; 90:4112-4116
Essex D W, Chen K, Swiatkowska M. Localisation of protein disulfide isomerase to the external surface of the platelet plasma membrane. Blood. 1995; 86:2168-2173
Chen K, Detwiler T C, Essex D W. Characterisation of protein disulphide isomerase released from activated platelets. Br. J. Haematol. 1995; 90:425-431
Chen K, Lin Y, Detwiler T C. Protein disulfide isomerase activity is released by activated platelets. Blood. 1992; 79:2226-2228
Burgess J K, Hotchkiss K A, Suter C, et al. Physical proximity and functional association of glycoprotein 1ba and protein disulfide isomerase on the platelet plasma membrane. J. Biol. Chem. 2000; 275:9758-9766
Essex D W, Li M, Miller A, Feinman R D. Protien disulfide isomerase and sulfhydryl-dependent pathways in platelet activation. Biochemistry. 2001; 40:6070-6075
Lahav J, Jurk K, Hess O, et al. Sustained integrin ligation involves extracellular free sulfhdryls and enzymatically catalyzed disulfide exchange. Blood. 2002; 100:2472-2478
Lahav J, Wijnen E M, Hess O, et al. Enzymatically catalyzed disuflide exchange is required for platelet adhesion to collagen via integrin a₂b₁. Blood. 2003; 102:2085-2092
Lahav J, Gofer-Dadosh N, Luboshitz J, Hess O, Shaklai M. Protein disulfide isomerase mediates integrin-dependent adhesion. FEBS. 2000; 475:89-92
Zai A, Rudd A, Scribner AW, Loscalzo J. Cell-surface protein disulfide isomerase catalyses transnitrosation and regulates intracellular transfer of nitric oxide. J. Clin. Invest. 1999; 103:393-399
Yan B, Smith J W. A redox site involved in integrin activation. J. Biol. Chem. 2000; 275:39964-39972
Jiang X-M, Fitzgerald M, Grant C M, Hogg P J. Redox control of exofacial protein thiols/disulfides by protein disuflide isomerase. J. Biol. Chem. 1999; 274:2416-2423
O'Neill S, Robinson A, Deering A, Ryan M, Fitzgerald D J, Moran N. The platelet integrin a_{IIb}b₃ has an endogenous thiol isomerase activity. J. Biol. Chem. 2000; 275:36984-36990
Gibbins J, Asselin J, Farndale R, Barnes M, Law C-L, Watson S P. Tyrosine phosphorylation of the Fc receptor g-chain in collagen-stimulated platelets. J. Biol. Chem. 1996; 271:18095-18099

Cicmil M, Thomas J M, Sage T, et al. Collagen, convulxin, and thrombin stimulate aggregation-independent tyrosine phosphorylation of CD31 in platelets: Evidence for the involvement of Src family kinases. J. Biol. Chem. 2000; 275:27339-27347

Pigiet V P, Schuster B J. Thioredoxin-catalyzed refolding of disulfide containing proteins. Proc. Nat'l Acad. Sci. USA. 1986; 83:7643-7647
Polgár J, Clemetson J M, Kehrel B E, et al. Platelet Activation and Signal Transduction by Convulxin, a C-type Lectin from Crotalus durissus terrificus (Tropical Rattlesnake) Venom via the p62/GPVI Collagen Receptor. J. Biol. Chem. 1997; 272:13576-13583
Kanaji S, Kanaji T, Furihata K, Kato K, Ware J L, Kunicki T J. Convulxin Binds to Native, Human Glycoprotein Ib. J. Biol. Chem. 2003; 278:39452-39460
Hayano T, Kikuchi M. Cloning and sequencing of the cDNA encoding human P5. Gene. 1995; 164:377-378
Kramer B, Ferrari D M, Klappa P, Pohlmann N, Soling H-D. Functional roles and efficiencies of the thioredoxin boxes of calcium-binding proteins 1 and 2 in protein folding. Biochem. J. 2001; 357:83-95
Jones K L, Hughan S C, Dopheide S M, Farndale R W, Jackson S P, Jackson D E. Platelet endothelial cell adhesion molecule-1 is a negative regulator of platelet-collagen interactions. Blood. 2001; 98:1456-1463
Cicmil M, Thomas J M, Leduc M, Bon C, Gibbins J M. Platelet endothelial cell adhesion molecule-1 signaling inhibits the activation of human platelets. Blood. 2002; 99:137-144
Shattil S J, Kashiwagi H, Pampori N. Integrin Signaling: The Platelet Paradigm. Blood. 1998; 91:2645-2657
D.R. Phillips, I. F. Charo, R. M. Scarborough. GPIIb-IIIa: The responsive integrin. Cell. 1991:359-362
Palabrica T, Lobb R, Furie B C, et al. Leukocyte accumulation promoting fibrin deposition is mediated in vivo by P-selectin on adherent platelets. Nature. 1992; 359:848-851
Furie B, Furie B C, Flaumenhaft R. A journey with platelet P-selectin: the molecular basis of granule secretion, signalling and cell adhesion. Thromb. Haemost. 2001; 86:214-221
Ren Z, Riley N J, Needleman L A, Sanders J M, Swanson G T, Marshall J. Cell surface expression of GluR5 Kainate receptors is regulated by an endoplasmic reticulum retention signal. J. Biol. Chem. 2003; 278:52700-52709
Vu T K, Hung D T, Wheaton V I, Coughlin S R. Molecular cloning of a functional thrombin receptor reveals a novel proteolytic mechanism of receptor activation. Cell. 1991; 64:1057-1068
Kahn M L, Zheng Y W, Huang W, et al. A dual thrombin receptor system for platelet activation. Nature. 1998; 394:690
Morton L F, Hargreaves P G, Farndale R W, Young R D, Barnes M J. Integrin α₂β₁-independent activation of platelets by simple collagen-like peptides: collagen tertiary (triple-helical) and quaternary (polymeric) structures are sufficient alone for alpha 2 beta1-independent platelet reactivity. Biochem. J. 1995; 306:337-344
Nieswandt B, Watson S P. Platelet-collagen interaction: is GP VI the central receptor? Blood. 2003; 102:449-461
Wang Z, Leisner T M, Parise L V. Platelet α₂β₁ integrin activation: contribution of ligand internalization and the 2-cytoplasmic domain. Blood. 2003; 102:1307-1315
Elton C M, Smethurst P A, Eggleton P, Farndale R W. Physical and functional interaction between cell-surface calreticulin and the collagen receptors integrin α₂β₁ and glycoprotein VI in human platelets. Thromb Haemost. 2002; 88:648-654

### EQUIVALENTS

From the foregoing detailed description of the invention, it should be apparent that the invention provides a method of treating a disease or condition mediated by platelet activation comprising supplying a therapeutically effective amount of an ERP5 inhibitor to a patient in need thereof, so as to reduce the effects of platelet activation in said patient. Although particular embodiments of the invention have been disclosed herein in detail, this has been done by way of example for purposes of illustration only, and is not intended to be limiting with respect to the scope of the appended claims which follow. In particular, it is contemplated by the inventor that substitutions, alterations, and modifications may be made to the invention without departing from the spirit and scope of the invention as defined by the claims.

What is claimed is:

1. A method of treating a disease or condition mediated by platelet activation comprising supplying a therapeutically effective amount of a ERP5 inhibitor to a patient in need thereof, so as to reduce the effects of platelet activation in said patient.

2. The method according to claim 1 wherein, said ERP5 inhibitor is an anti-ERP5 antibody.

3. The method according to claim 1 wherein, the effect on platelet activation which is reduced is platelet aggregation.

4. The method according to claim 1 wherein, the disease to be treated is thrombosis.

5. A method of modulating platelet activation comprising contacting an effective amount of an ERP5 inhibitor with a population of platelets so as to modulate activation in response to exposure to platelet agonists.

6. The method according to claim 5 wherein, the ERP5 inhibitor is an anti-ERP5 antibody.

7. The method according to claim 5 wherein, the response to platelet activation which is modulated is platelet aggregation.

8. The method according to claim 5 wherein, the effect is a reduction in platelet aggregation.

9. The method according to claim 8 wherein there is a reduced risk of thrombosis.

10. The method according to claim 5 wherein, the modulation effect is a reduction in fibrinogen binding activity.

11. A pharmaceutical composition for reducing platelet activation in response to at least one platelet agonist comprising an ERP5 inhibitor and at least one pharmaceutically acceptable diluent or excipient.

12. The composition according to claim 11 wherein, the ERP5 inhibitor is an anti-ERP5 antibody.

13. An anti-human ERP5 antibody which specifically binds to platelet surface ERP5 so as to inhibit the effects of platelet activation in response to exposure to a platelet agonist.

14. The anti-human ERP5 antibody according to claim 13 wherein, platelet aggregation is inhibited.

15. The anti-human ERP5 antibody according to claim 13 wherein, interaction of ERP5 with integrin α_{iib}β₃ is inhibited.

16. The anti-human ERP5 antibody according to claim 13 wherein, fibrinogen binding is inhibited.

17. A method for identifying a molecule capable of inhibiting ERP5 modulated activation of platelets comprising the steps of:
a) exposing ERP5 to the molecule of interest;
b) determining the thiol isomerase activity of the ERP5;
c) exposing platelets to those molecules which inhibit ERP5 thiol isomerase activity;
d) activating said platelets by exposure to a platelet agonist;
e) determining the level of platelet aggregation in response to platelet activation; and
f) comparing the level of aggregation with platelets not exposed to the molecule of interest.

18. The method as claimed in claim 17 wherein, the thiol isomerase activity is determined by the ability of ERP5 to renature RNAse.

19. The method as claimed in claim 17 wherein, the platelets are activated by exposure to collagen, convulxin or thrombin.

20. The method as claimed in claim 17 wherein, aggregation is determined using flow cytometry.

21. A method of making a pharmaceutical composition for the treatment of thrombosis, comprising combining an inhibitor identified according to the method of claim 17 together with a pharmaceutically acceptable diluent or excipient.
